(19) United States
(12) Patent Application Publication
(10) Pub. No.: US 2012/0296211 A1
(43) Pub. Date: Nov. 22, 2012

(54) ULTRASOUND DIAGNOSTIC APPARATUS

(75) Inventor: Atsushi OSAWA, Kanagawa (JP)

(73) Assignee: FUJIFILM CORPORATION, Tokyo (JP)

(21) Appl. No.: 13/473,233

(22) Filed: May 16, 2012

(30) Foreign Application Priority Data
May 17, 2011 (JP) 2011-110342

(51) Int. Cl. A61B 8/14; A61N 5/06

(52) U.S. Cl. 600/439

(57) ABSTRACT

An ultrasound diagnostic apparatus includes a light irradiator for irradiating a lattice point set in the region to be imaged with light converging to the lattice point, so as to impart heat locally to the point; an image for distortion amount calculation-generating section for generating an ultrasound image for distortion amount calculation based on a reception signal of ultrasound generated; and a distortion amount calculating section for calculating the difference between the position of the lattice point on an ultrasound image for distortion amount calculation and the absolute coordinates of the lattice point as the distortion amount. Such apparatus allows precise sound speed values in the living body to be obtained and, accordingly, an ultrasound image of high accuracy to be taken. In consequence, a more accurate diagnosis is conducted on the region to be diagnosed in a subject.

### BACKGROUND OF THE INVENTION

The present invention relates to ultrasound diagnostic apparatus generating an ultrasound image for diagnosis by transmitting and receiving ultrasound to image an organ or the like in the living body.

In the medical field, ultrasound diagnostic apparatus employing ultrasound images have already been put to practical use. A typical ultrasound diagnostic apparatus for medical use has an ultrasound probe with a transducer array built therein and an apparatus body connected with the ultrasound probe, and generates an ultrasound image by transmitting ultrasound from the ultrasound probe toward a subject, receiving an ultrasonic echo from the subject on the ultrasound probe, and electrically processing a reception signal corresponding to the received echo in the apparatus body.

During the generation of an ultrasound image in an ultrasound diagnostic apparatus, it is assumed that the sound speed is constant in the living body as a subject. In fact, the sound speed varies in value in the living body, causing spatial distortion in an ultrasound image.

In a recent attempt to conduct a more accurate diagnosis on the region to be diagnosed in a subject, the sound speed is measured in the region to be diagnosed to thereby correct such image distortion.

Measurement in various regions, such as of vascular wall thickness or tumor size, is improved in accuracy by correcting distortion of an ultrasound image.

As an example, JP 2010-99452 A has proposed the ultrasound diagnostic apparatus in which a plurality of lattice points are set in the vicinity of the region to be diagnosed, and an arithmetical operation for local sound speed values is performed based on reception data obtained by transmitting and receiving an ultrasonic beam to and from each lattice point.

JP 2009-279306 A has proposed the ultrasound diagnostic apparatus in which the degree of beam focusing in focusing processing is determined with respect to a plurality of first regions, and sound speed values are obtained with respect to the individual first regions, and also with respect to a plurality of second regions provided by dividing the first regions into smaller ones.

### SUMMARY OF THE INVENTION

The ultrasound diagnostic apparatus as described in JP 2010-99452 A and in JP 2009-279306 A are each capable of obtaining local sound speed values in the living body by transmitting an ultrasonic beam from an ultrasound probe toward the inside of a subject and receiving an ultrasonic beam returning from the subject, which makes it possible to indicate information of local sound speed values on, for instance, a B-mode image in a superimposed manner.

When an ultrasonic beam is transmitted toward the set lattice points or region in order to obtain local sound speed values, however, the ultrasonic beam may be transmitted to a location deviated from the set lattice points or region because a precise sound speed is unknown, so that it is not possible to properly obtain local sound speed values.

An object of the present invention is to provide an ultrasound diagnostic apparatus capable of obtaining precise sound speed values in the living body and, accordingly, taking an ultrasound image of high accuracy, allowing a more accurate diagnosis to be conducted on the region to be diagnosed in a subject.

Another object of the present invention is to provide an ultrasound diagnostic apparatus simplifying tissue characterization, determination of the progression of hepatic cirrhosis or fatty liver for instance, by obtaining precise sound speed values in the living body.

In order to achieve the above objects, the present invention provides an ultrasound diagnostic apparatus comprising: an ultrasound probe including a transducer array for transmitting ultrasound, receiving an ultrasonic echo reflected from a subject and outputting a reception signal in response to ultrasound received; and a diagnostic apparatus body including an image generating section for generating an ultrasound image for diagnosis in accordance with the reception signal as outputted from the transducer array, the apparatus further comprising: a lattice point setting unit for setting lattice points in a region to be imaged; a light irradiator for irradiating each of the lattice points as set by the lattice point setting unit with light converging thereto, to thereby impart heat locally to the irradiated lattice point; an image for distortion amount calculation-generating section for generating an ultrasound image for distortion amount calculation based on a reception signal outputted from the transducer array having received ultrasound resulting from irradiation of an inside of the subject with light by the light irradiator; a lattice point detector for detecting a position of the irradiated lattice point on the ultrasound image for distortion amount calculation; and a distortion amount calculating section for calculating, as a distortion amount, difference between absolute coordinates of the lattice point as irradiated with light by the light irradiator and the position of the lattice point that is detected on the ultrasound image for distortion amount calculation.

It is preferable that the transducer array transmits ultrasound to the subject during the irradiation of the inside of the subject with light by the light irradiator; the transducer array receives not only ultrasound resulting from the irradiation of the inside of the subject with light by the light irradiator but an ultrasonic echo resulting from transmission of ultrasound to the subject by the transducer array, so as to output a reception signal; and the image for distortion amount calculation-generating section generates the ultrasound image for distortion amount calculation based on a reception signal outputted from the transducer array.

It is preferable that the ultrasound diagnostic apparatus further comprises an image corrector for correcting the ultrasound image for diagnosis using the distortion amount calculated by the distortion amount calculating section.

The light irradiator is preferably provided on the ultrasound probe.

It is preferable that the ultrasound diagnostic apparatus further comprises a sound speed map generating section for generating a sound speed map in the subject, and the sound speed map generating section uses the distortion amount calculated by the distortion amount calculating section to correct the reception signal assigned to sound speed map generation, so as to generate the sound speed map.

It is preferable that the image generating section for generating an ultrasound image generates the ultrasound image using the sound speed map as generated by the sound speed map generating section.

Preferably, the sound speed map as generated by the sound speed map generating section is displayed in such a manner that it is superimposed on the ultrasound image as generated by the image generating section.

The light irradiator preferably has a light source array including a plurality of light sources, and a microlens array including a plurality of microlenses associated with the light sources of the light source array, respectively.

According to the ultrasound diagnostic apparatus of the present invention that has such configuration as above, that is to say, comprises a lattice point setting unit for setting a plurality of lattice points in a region to be imaged; a light irradiator for irradiating each of the lattice points as set by the lattice point setting unit with light converging thereto, to thereby impart heat locally to the irradiated lattice point; an image for distortion amount calculation-generating section for generating an ultrasound image for distortion amount calculation based on a reception signal outputted from the transducer array having received ultrasound resulting from irradiation of an inside of the subject with light by the light irradiator; a lattice point detector for detecting a position of the irradiated lattice point on the ultrasound image for distortion amount calculation; and a distortion amount calculating section for calculating, as a distortion amount, difference between absolute coordinates of the lattice point as irradiated with light by the light irradiator and the position of the lattice point that is detected on the ultrasound image for distortion amount calculation, it is possible to take an ultrasound image of high accuracy, and conduct a more accurate diagnosis on the region to be diagnosed in a subject. In addition, it is possible to obtain precise sound speed values in the living body, and simplify tissue characterization, measurement of the progression of hepatic cirrhosis or fatty liver for instance, by obtaining precise sound speed values in the living body.

### BRIEF DESCRIPTION OF THE DRAWINGS

In the accompanying drawings:

FIG. 1 is a block diagram illustrating a conceptual configuration of an embodiment of the ultrasound diagnostic apparatus according to the present invention;

FIG. 2 is a diagram schematically showing lattice points and a light irradiator in the ultrasound diagnostic apparatus of FIG. 1;

FIG. 3 is a diagram schematically showing the positions of lattice points and an ultrasound image for distortion amount calculation in the ultrasound diagnostic apparatus of FIG. 1;

FIG. 4 is a diagram schematically showing another exemplary light irradiator;

FIGS. 5A and 5B are diagrams schematically illustrating the principle of sound speed operation; and

FIG. 6 is a block diagram illustrating a conceptual configuration of another embodiment of the ultrasound diagnostic apparatus according to the present invention.

### DETAILED DESCRIPTION OF THE INVENTION

In the following, the ultrasound diagnostic apparatus of the present invention is detailed in reference to the preferred embodiments as shown in the accompanying drawings.

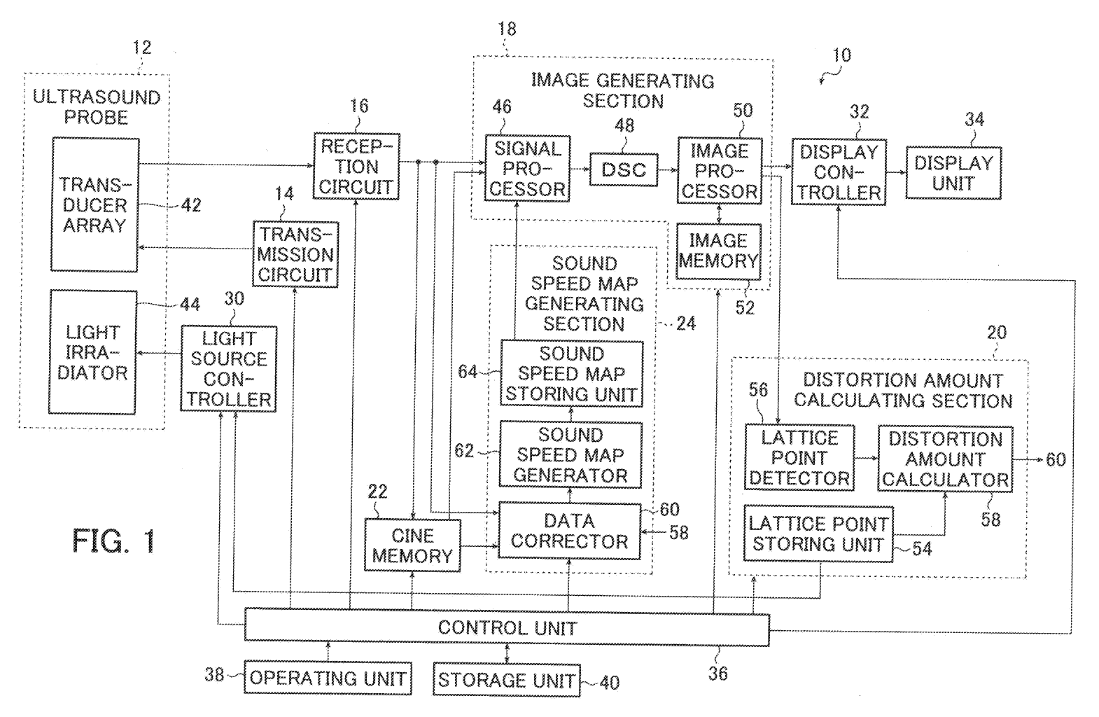

FIG. 1 is a block diagram illustrating a conceptual configuration of an embodiment of the ultrasound diagnostic apparatus according to the present invention.

An ultrasound diagnostic apparatus 10 includes an ultrasound probe 12, a transmission circuit 14 and a reception circuit 16 each connected with the ultrasound probe 12, an image generating section 18, a distortion amount calculating section 20, a cine memory 22, a sound speed map generating section 24, a light source controller 30, a display controller 32, a display unit 34, a control unit 36, an operating unit 38, and a storage unit 40.

In the ultrasound diagnostic apparatus 10 as shown that is adapted to take an ultrasound image for diagnosis and generate a sound speed map, an ultrasound image for distortion amount calculation is also taken to calculate the distortion amount of the ultrasound image for diagnosis, and the ultrasound image for diagnosis and the sound speed map are corrected using the distortion amount as calculated.

The ultrasound probe 12 has a transducer array 42 for use in conventional ultrasound diagnostic apparatus, and a light irradiator 44 used for the calculation of the distortion amount of an ultrasound image for diagnosis.

The transducer array 42 includes a plurality of ultrasound transducers one- or two-dimensionally arranged. During the taking of ultrasound images for diagnosis and for distortion amount calculation, the ultrasound transducers each transmit ultrasound in response to the driving signal as fed from the transmission circuit 14, and receive ultrasound resulting from the light irradiation by the light irradiator 44 and an ultrasonic echo from the subject so as to output reception signals.

The processing performed by the transducer array 42, the transmission circuit 14, the reception circuit 16 and the image generating section 18 does not differ between the cases where an ultrasound image for distortion amount calculation is taken and where a normal ultrasound image for diagnosis is taken. For this reason, the taking of an ultrasound image for diagnosis will basically be illustrated in the following as long as it is not necessary to distinguish an ultrasound image for distortion amount calculation from an ultrasound image for diagnosis.

Each ultrasound transducer is comprised of a vibrating element having a piezoelectric body and electrodes formed at both ends of the piezoelectric body, with examples of the material for the body including a piezoelectric ceramic typified by lead zirconate titanate (PZT), a polymeric piezoelectric material typified by polyvinylidene fluoride (PVDF), and a piezoelectric single crystal typified by lead magnesium niobate-lead titanate solid solution (PMN-PT).

If a pulsed voltage or a continuous wave voltage is applied across the electrodes of the vibrating element as above, the piezoelectric body expands and contracts, and ultrasound in pulse or continuous wave form is generated from the vibrating element. Ultrasounds generated from the individual vibrating elements are synthesized into an ultrasonic beam. In addition, each vibrating element expands and contracts during the reception of propagating ultrasound to generate an electric signal, and the electric signal is outputted as a reception signal representing the reception of ultrasound.

When an ultrasound image for distortion amount calculation is taken, the light irradiator 44 emits light focused on a specified position in the subject, concurrently with the action of the transducer array 42 under the control of the light source controller 30.

The light irradiator 44 will be detailed later.

The transmission circuit 14 includes a plurality of pulsers, for instance, and is adapted to modify, based on the transmission delay pattern as selected in response to a control signal from the control unit 36, the delay amounts of the driving signals to be fed to the ultrasound transducers of the transducer array 42 so that ultrasounds transmitted from the ultrasound transducers may form an ultrasonic beam, and then feed the ultrasound transducers with their respective driving signals.

The reception circuit 16 amplifies the reception signals as transmitted from the individual ultrasound transducers of the transducer array 42 to subject them to analog/digital conversion, then provides the reception signals with their respective delays in accordance with a sound speed or sound speed distribution set on the basis of the reception delay pattern as selected in response to a control signal from the control unit 36, and adds the delayed signals to thereby perform reception focusing. The reception focusing allows reception data (sound ray signal) to be obtained from a well-focused ultrasonic echo.

The reception circuit 16 feeds the reception data to the image generating section 18, a data corrector 60 of the sound speed map generating section 24, and the cine memory 22.

The image generating section 18 generates an ultrasound image for diagnosis (and an ultrasound image for distortion amount calculation) from the reception data as fed from the reception circuit 16.

The image generating section 18 has a signal processor 46, a digital scan converter 48, an image processor 50, and an image memory 52.

The signal processor 46 uses a sound speed map stored in a sound speed map storing unit 64 of the sound speed map generating section 24 described later to correct the reception data as generated by the reception circuit 16 for attenuation due to distance in accordance with the depth of the position where ultrasound was reflected, and subjects the corrected data to envelope demodulation so as to generate a B-mode image signal as tomographic image information on a tissue in the subject.

The digital scan converter (DSC) 48 subjects the B-mode image signal as generated by the signal processor 46 to the conversion (raster conversion) into an image signal compatible with the conventional television signal scanning method.

The image processor 50 subjects the B-mode image signal as inputted from the DSC 48 to various types of image processing, such as grayscaling, as required, then outputs the B-mode image signal to the display controller 32 or stores it in the image memory 52 in the case of a normal ultrasound image for diagnosis.

In the case of an ultrasound image for distortion amount calculation, the B-mode image signal is fed from the image processor 50 to a lattice point detector 56.

The light irradiator 44 of the ultrasound probe 12 is so positioned as to be adjacent to the transducer array 42, and emits the light which is focused on a specified position in the region (scan area) where the transducer array 42 performs ultrasound transmission/reception.

Specifically, the light irradiator 44 emits, under the control of the light source controller 30, the light which is focused on the position of a lattice point read from a lattice point storing unit 54 described later.

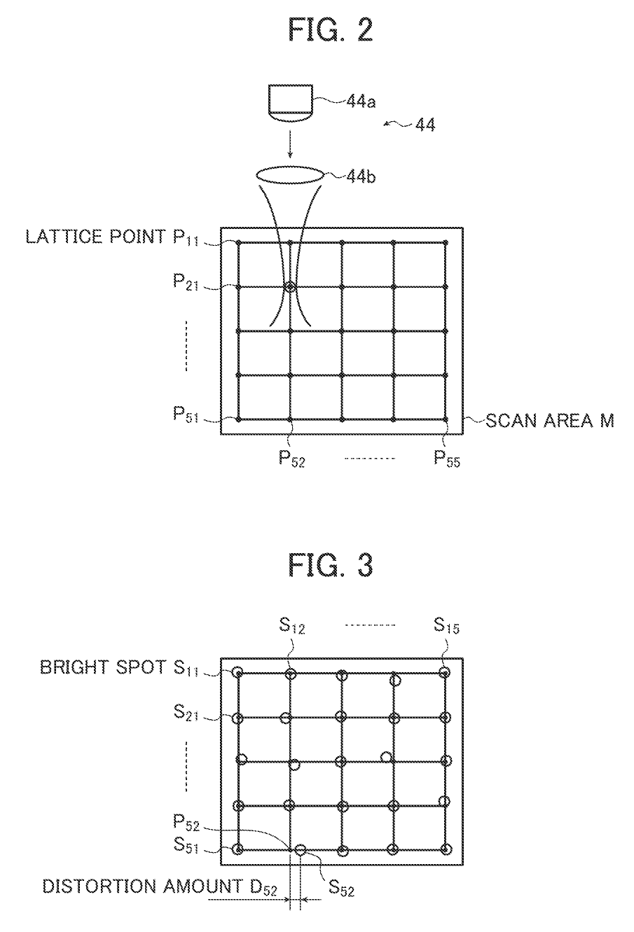

FIG. 2 is a schematic diagram showing lattice points P set and a scan area M, and also showing for convenience the light irradiator 44 in association with the scan area M. In the example as shown, the light irradiator 44 has a light source 44a and a lens 44b, and emits light focused on the positions of the lattice points P by focusing the light as emitted from the light source 44a with the lens 44b.

In FIG. 2, the lattice points P as set are arranged in five rows and five columns, with a lattice point in the ith row and the jth column being denoted by Pᵢⱼ.

The light irradiator 44 is displaced by a displacement means not shown, so as to cause it to irradiate the position of the lattice point Pᵢⱼ with light.

If the light irradiator 44 irradiates the inside of a subject with light to thereby impart heat to the irradiated region, cubical expansion due to the heat occurs in the region, leading to the generation of ultrasound.

When an ultrasound image for distortion amount calculation is taken, the transducer array 42 transmits an ultrasonic beam to the inside of a subject and the light irradiator 44 irradiates the inside of the subject with light in accordance with the instruction from the control unit 36, so that the transducer array 42 receives not only an ultrasonic echo derived from the ultrasonic beam from the transducer array 42 in itself but ultrasound resulting from the light irradiation by the light irradiator 44, so as to output a reception signal. An ultrasound image for distortion amount calculation is generated from the reception signal thus outputted.

FIG. 3 schematically shows an ultrasound image for distortion amount calculation and the lattice point Pᵢⱼ. If the position of the lattice point Pᵢⱼ is irradiated with light by the light irradiator 44, the position as irradiated with light is displayed on the ultrasound image for distortion amount calculation as a bright spot Sᵢⱼ having a high brightness, as shown in FIG. 3. The bright spots Sᵢⱼ of FIG. 3 are arranged in five rows and five columns, and are corresponding to the lattice points Pᵢⱼ as arranged in five rows and five columns.

While the sound speed varies with region in the living body, the speed of light is almost constant in the living body. Consequently, the position of the lattice point Pᵢⱼ is irradiated with light by the light irradiator 44 in an accurate manner.

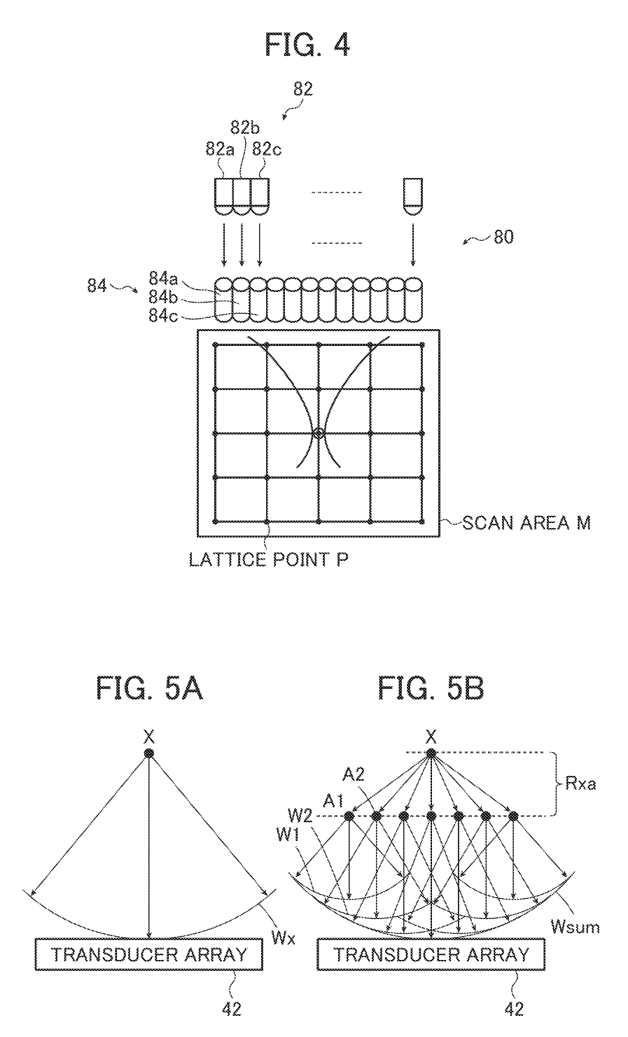

In the example as shown, the light irradiator 44 has one light source 44a and one lens 44b, and is displaced, that is to say, its light source 44a and lens 44b are displaced so as to change the position to be irradiated with light, although the present invention is not limited thereto. The light irradiator 44 may have such a configuration as of a light irradiator 80 shown in FIG. 4, which has a light source array 82 comprised of a plurality of light sources 82a, 82b, 82c, and so forth, and a microlens array 84 comprised of a plurality of microlenses 84a, 84b, 84c, and so forth, and focuses light on a specified position by shifting the light sources in light-emitting timing.

The light source controller 30 reads the positions of the lattice points P as stored in the lattice point storing unit 54, in accordance with the instruction from the control unit 36, and controls the light irradiator 44 to sequentially irradiate the positions of the lattice points P with light.

The distortion amount calculating section 20 calculates, under the control of the control unit 36, the distortion amount of an ultrasound image taken for diagnosis.

The distortion amount calculating section 20 includes the lattice point storing unit 54, the lattice point detector 56, and a distortion amount calculator 58.

The lattice point storing unit 54 stores the lattice points P set in the scan area M where the transducer array 42 performs ultrasound transmission/reception.

In FIG. 2, the lattice points Pᵢⱼ are set at intersections of five horizontal lines and five vertical lines in a plane perpendicular to the light irradiation direction of the light irradiator 44.

The lattice points P may be specified in position and number previously in accordance with imaging conditions and the like, or set by an operator through the operating unit 38.

The number of the lattice points P to be set is not particularly limited as long as local sound speed values are precisely calculated and, consequently, an ultrasound image of high accuracy is generated.

The lattice point detector 56 detects the position of the bright spot Sᵢⱼ in an ultrasound image for distortion amount calculation fed from the image processor 50.

The method of detecting the bright spot Sᵢⱼ is not particularly limited, and various known methods including that using a threshold to detect a position with a high brightness are usable.

The lattice point detector 56 feeds information on the position of the detected bright spot Sᵢⱼ to the distortion amount calculator 58.

The distortion amount calculator 58 compares the position of the lattice point Pᵢⱼ as read from the lattice point storing unit 54 with the information on the position of the bright spot Sᵢⱼ as fed from the lattice point detector 56, and calculates the deviation between the positions of the lattice point Pᵢⱼ and of the bright spot Sᵢⱼ as a distortion amount Dᵢⱼ, with the distortion amount D being determined for each lattice point P.

Since the speed of light is almost constant in the living body as mentioned before, the light irradiator 44 is able to irradiate the position of the lattice point Pᵢⱼ with light in an accurate manner. On the other hand, ultrasound is generated in the position of the lattice point Pᵢⱼ that is irradiated with light. When the generated ultrasound is received to generate an ultrasound image for distortion amount calculation, the position as irradiated with light is displayed on the image as the bright spot Sᵢⱼ.

In this regard, the bright spot Sᵢⱼ is displayed on the image in a position different from the position of the lattice point Pᵢⱼ if the local sound speed value to be used for the generation of the ultrasound image is different from an actual sound speed value in the living body. This deviation between the lattice point Pᵢⱼ and the bright spot Sᵢⱼ is determined by the distortion amount calculator 58 as the distortion amount Dᵢⱼ.

A precise local sound speed value can be obtained by determining the distortion amount Dᵢⱼ with respect to the lattice point Pᵢⱼ and the bright spot Sᵢⱼ, and correcting a local sound speed value in the living body with the distortion amount Dᵢⱼ when the sound speed value is obtained in the sound speed map generating section 24.

The distortion amount calculator 58 feeds the calculated distortion amount Dᵢⱼ to the data corrector 60 of the sound speed map generating section 24.

The cine memory 22 sequentially stores reception data outputted from the reception circuit 16. In addition, the cine memory 22 associates frame rate-related information (e.g., the depth of the position where ultrasound was reflected, the scan line density, a parameter indicating the width of visual field) inputted from the control unit 36 with the reception data to store the information as such.

The sound speed map generating section 24 obtains local sound speed values in different positions in a subject to generate a sound speed map under the control of the control unit 36.

The sound speed map generating section 24 includes the data corrector 60, a sound speed map generator, and a sound speed map storing unit 64.

The data corrector 60 reads the reception data as stored in the cine memory 22, and corrects the reception data for position-related information (information on the position where ultrasound was reflected, and the like) using the distortion amount Dᵢⱼ as fed from the distortion amount calculator 58, so as to generate the corrected reception data.

The method for position correction performed by the data corrector 60 is not particularly limited, and available methods include various position correcting methods used for the image processing in ultrasound diagnostic apparatus, such as nearest neighbor interpolation, linear/quadratic/cubic interpolation, polynomial interpolation, Lagrange interpolation, and spline interpolation.

The data corrector 60 having generated the corrected reception data feeds the corrected data to the sound speed map generator 62.

The sound speed map generator 62 uses the corrected reception data as fed from the data corrector 60 to perform operation for local sound speed values in the tissue to be diagnosed in a subject, so as to generate a sound speed map.

The method for operation performed by the sound speed map generator 62 for local sound speed values is not particularly limited, and available methods include the method as described in JP 2010-99452 A which was filed by the present applicant.

The method of JP 2010-99452 A is explained in reference to FIGS. 5A and 5B. If ultrasound is transmitted to the inside of a subject, a receiving wave Wx from a lattice point X as a reflection point on the subject reaches the transducer array 42 as shown in FIG. 5A. With a plurality of lattice points A1, A2, and so forth at regular intervals being positioned more shallowly, that is to say, closer to the transducer array 42 than the lattice point X, a composite wave Wsum of receiving waves W1, W2, and so forth from the lattice points A1, A2, and so forth each having received a receiving wave from the lattice point X is identical to the receiving wave Wx from the lattice point X according to Huygens' principle, as shown in FIG. 5B. In the above method, a local sound speed value at the lattice point X is obtained based on the fact as above.

Initially, the optimal sound speed value is obtained for each of the lattice points X as well as A1, A2, and so forth. The optimal sound speed value refers to a sound speed value allowing an ultrasound image with the highest contrast and sharpness when ultrasound images are taken by performing focusing calculation for each lattice point based on the sound speeds as differently specified. The optimal sound speed value may be determined based on the contrast of an image, the spatial frequency or dispersion in a scanning direction or the like, as described in JP 8-317926 A for instance.

The optimal sound speed value for the lattice point X is used to calculate the waveform of a virtual receiving wave Wx from the lattice point X.

In addition, a hypothetical local sound speed value V at the lattice point X is variously changed, and a virtual composite wave Wsum of the receiving waves W1, W2, and so forth from the lattice points A1, A2, and so forth is calculated for each value V. In this regard, it is assumed that, in a region Rxa between the lattice point X and the lattice points A1, A2, and so forth, the sound speed is constant and equal to the local sound speed value at the lattice point X. The times to be taken by ultrasound propagating from the lattice point X to reach the lattice points A1, A2, and so forth are XA1/V, XA2/V, and so forth, respectively, with XA1, XA2, and so forth referring to the distances between the lattice points A1, A2, and so forth and the lattice point X, respectively. The virtual composite wave Wsum is obtained by synthesizing reflected waves sent from the lattice points A1, A2, and so forth with their respective delays of XA1/V, XA2/V, and so forth.

Next, with respect to the virtual composite waves Wsum as calculated by variously changing the hypothetical local sound speed value V at the lattice point X, their respective errors from the virtual receiving wave Wx from the lattice point X are calculated, and the hypothetical local sound speed value V which allows the minimal error is determined as the local sound speed value at the lattice point X. The error of a virtual composite wave Wsum from the virtual receiving wave Wx from the lattice point X may be calculated by the method in which cross correlation is obtained between the two waves, the method in which phasing/adding is carried out by multiplying the receiving wave Wx by a delay obtained from the composite wave Wsum, or the method in which phasing/adding is carried out by multiplying the composite wave Wsum by a delay obtained from the receiving wave Wx.

Based on the corrected reception data as generated by the data corrector 60, operation for local sound speed values in individual parts in a subject is performed as described above so as to generate a sound speed map in the subject.

As mentioned before, in the method in which sound speed values (local sound speed values) in the positions of individual regions (lattice points) in the living body are obtained based on reception data obtained by transmitting an ultrasonic beam to the individual lattice points and receiving an ultrasonic echo derived from the transmitted beam, the ultrasonic beam to be transmitted to the individual lattice points may be transmitted to a location deviated from the set lattice points because local sound speed values in the living body are unknown. Accordingly, it is not possible to obtain precise sound speed values.

In contrast, according to the present invention, irradiation with light is performed by the light irradiator 44, the deviation between the position of a bright spot obtained from ultrasound resulting from the irradiation with light and the position of the lattice point P as irradiated with light is calculated as the distortion amount D, and reception data for the calculation of local sound speed values is corrected using the distortion amount D. The reception data thus corrected allows precise local sound speed values (sound speed map) even if actual sound speed values are unknown and, as a consequence, an ultrasonic beam has been transmitted to a location deviated from accurate positions of the set lattice points or region.

With precise sound speed values in the living body being obtained, tissue characterization, determination of the progression of hepatic cirrhosis or fatty liver for instance, can be simplified.

The signal processor 46, as performing various processes on the reception data as fed from the reception circuit 16 using an accurate sound speed map, is able to generate the B-mode image signal (ultrasound image for diagnosis) of high accuracy which has been corrected for distortion due to the variability in sound speed. A more accurate diagnosis can be conducted on the region to be diagnosed in a subject by taking ultrasound images of high accuracy.

Use of an accurate sound speed map for the generation of ultrasound images will complete the conditions for ultrasonic echo combination, so that the sensitivity is improved over the entire region to be imaged, and the resolution is also improved.

The sound speed map storing unit 64 stores the sound speed map as generated by the sound speed map generator 62. The sound speed map storing unit 64 stores a specified sound speed as a sound speed map until a sound speed map is fed from the sound speed map generator 62.

The sound speed map storing unit 64 feeds a sound speed map to the signal processor 46 in accordance with the instruction from the control unit 36.

The display controller 32 causes the display unit 34 to display an ultrasound image for diagnosis, based on the B-mode image signal as subjected to image processing by the image processor 50.

The display unit 34 includes a display device such as LCD, and displays an ultrasound image under the control of the display controller 32.

The ultrasound diagnostic apparatus 10 may have two or more display modes, with a desired image being displayed on the display unit 34 by selecting a suitable display mode. For instance, the apparatus 10 may have the mode in which an ultrasound image (B-mode image) is solely displayed, the mode in which a B-mode image is displayed with a sound speed map superimposed thereon (in such a manner that the color or brightness is changed with local sound speed value, or points having the same local sound speed value are connected with one another by lines, for instance), and the mode in which a B-mode image and an image of sound speed map are displayed in parallel, and an operator may select any of the three display modes through the operating unit 38.

The control unit 36 controls the individual components of the ultrasound diagnostic apparatus 10 based on the instruction as inputted by an operator through the operating unit 38.

The operating unit 38 is used by an operator to perform input operations, and may be comprised of a keyboard, a mouse, a trackball, a touch panel, and the like.

The storage unit 40 is adapted to store operational programs and so forth, and such recording media as a hard disk, a flexible disk, MO, MT, RAM, CD-ROM, and DVD-ROM are available for the unit 40.

While the signal processor 46, the DSC 48, the image processor 50, the display controller 32, the sound speed map generating section 24, the lattice point detector 56 and the distortion amount calculator 58 are implemented by a CPU associated with operational programs for giving the CPU instructions on various kinds of processing, the above components may also be implemented by a digital circuitry.

Actions of the ultrasound diagnostic apparatus 10 are described below.

Actions during the calculation of the distortion amount Dᵢⱼ are as follows.

An operator brings the ultrasound probe 12 into contact with the surface of a subject. The ultrasound probe 12 as such transmits an ultrasonic beam from the transducer array 42 in response to a driving signal fed from the transmission circuit 14. At the same time, the light irradiator 44 irradiates the positions of lattice points set in advance with light under the control of the light source controller 30. The transducer array 42 receives an ultrasonic echo from the subject and ultrasound resulting from the light irradiation by the light irradiator 44 to output a reception signal.

The reception circuit 16 generates reception data from the reception signal as outputted from the transducer array 42, and feeds the data to the image generating section 18. In the image generating section 18, the signal processor 46 generates a B-mode image signal from the reception data, the DSC 48 subjects the B-mode image signal to raster conversion, and the image processor 50 subjects the signal to image processing to generate an ultrasound image for distortion amount calculation.

The ultrasound image for distortion amount calculation is fed to the lattice point detector 56 of the distortion amount calculating section 20 so as to detect the bright spot Sᵢⱼ. Information on the detected bright spot Sᵢⱼ is fed to the distortion amount calculator 58, then the distortion amount which is the deviation between the lattice point Pᵢⱼ as stored in the lattice point storing unit 54 and the detected bright spot Sᵢⱼ, is calculated. The calculated distortion amount Dᵢⱼ is fed to the data corrector 60 of the sound speed map generating section 24.

Actions during the taking of an ultrasound image for diagnosis and the generation of a sound speed map are as follows.

An operator brings the ultrasound probe 12 into contact with the surface of a subject. The ultrasound probe 12 as such transmits an ultrasonic beam from the transducer array 42 in response to a driving signal fed from the transmission circuit 14, and receives an ultrasonic echo from the subject so as to output a reception signal.

The reception circuit 16 generates reception data from the reception signal, and feeds the data to the cine memory 22 and the data corrector 60 of the sound speed map generating section 24. The data corrector 60 corrects the fed reception data with the distortion amount Dᵢⱼ to generate the corrected reception data. The sound speed map generator 62 performs operation for local sound speed values in individual parts in a subject based on the corrected reception data, so as to generate a sound speed map and feed the map to the sound speed map storing unit 64.

The reception circuit 16 also feeds the reception data to the image generating section 18. The signal processor 46 of the image generating section 18 reads the sound speed map as stored in the sound speed map storing unit 64 to process the reception data, and generates a B-mode image signal. The B-mode image signal is subjected to raster conversion by the DSC 48, then to image processing by the image processor 50 so as to generate an ultrasound image for diagnosis. The ultrasound image thus generated is stored in the image memory 52, and displayed on the display unit 34 by the display controller 32. Upon display of the ultrasound image, the sound peed map may be displayed along with the ultrasound image in accordance with the mode as selected by the operator.

As described above, in the ultrasound diagnostic apparatus 10 according to the present invention, the sound speed map generator 62 uses the reception data as corrected with the calculated distortion amount Dᵢⱼ to obtain local sound speed values, which allows precise local sound speed values (accurate sound speed map).

In addition, the sound speed map which is used by the signal processor 46 to process the reception data so as to generate a B-mode image signal has been corrected with the calculated distortion amount Dᵢⱼ, so that an ultrasound image of high accuracy is generated with no distortion. A more accurate diagnosis can be conducted on the region to be diagnosed in a subject by taking ultrasound images of high accuracy.

With precise sound speed values in the living body being obtained, tissue characterization, determination of the progression of hepatic cirrhosis or fatty liver for instance, can be simplified.

In the embodiment as described above, an accurate sound speed map is obtained by the correction with the distortion amount Dᵢⱼ before an ultrasound image is taken using the sound speed map, although the present invention is not limited thereto. It is also possible that the ultrasound image to be corrected is initially taken and stored, then an ultrasound image for distortion amount calculation is taken, the distortion amount Dᵢⱼ is calculated to obtain an accurate sound speed map, and the sound speed map as obtained is used to reconstitute the stored ultrasound image, so as to generate an ultrasound image of high accuracy with no distortion.

In the embodiment as described above, the image generating section 18 generates both ultrasound images for diagnosis and for distortion amount calculation, although the present invention is not limited thereto. The inventive apparatus may include an image generating section for generating an ultrasound image for diagnosis and an image generating section for generating an ultrasound image for distortion amount calculation separately from each other.

In the embodiment as described above, the calculated distortion amount Dᵢⱼ is used to correct local sound speed values, although the present invention is not limited thereto. The distortion amount Dᵢⱼ may also be used to correct an ultrasound image.

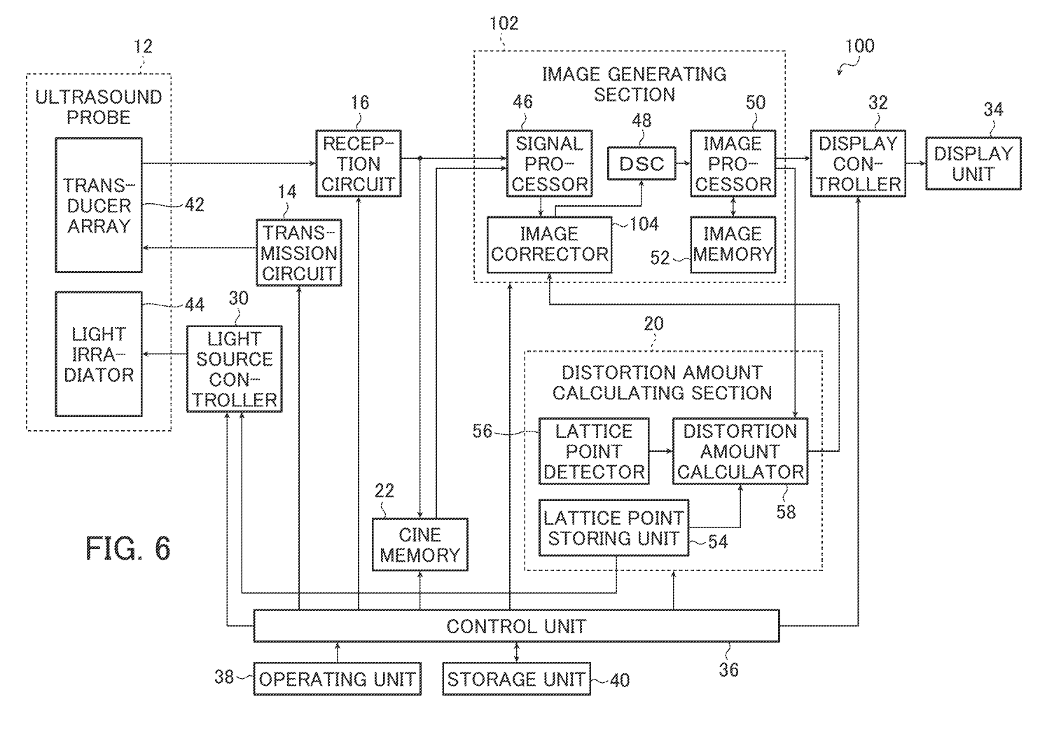

FIG. 6 is a block diagram illustrating a conceptual configuration of another embodiment of the ultrasound diagnostic apparatus of the present invention.

An ultrasound diagnostic apparatus 100 shown in FIG. 6 has the same configuration as the ultrasound diagnostic apparatus 10 of FIG. 1 except for the absence of the sound speed map generating section 24 and the replacement of the image generating section 18 by an image generating section 102 including an image corrector 104, so that like components are denoted by like reference characters, and the following description is chiefly made on differences in configuration.

The image generating section 102 includes the signal processor 46, the image corrector 104, the DSC 48, the image processor 50, and the image memory 52.

The image corrector 104 corrects the B-mode image signal as generated by the signal processor 46 for position-related information using the distortion amount Dᵢⱼ as calculated by the distortion amount calculator 58.

The method for position correction performed by the image corrector 104 is not particularly limited, and available methods include various known position correcting methods.

The image corrector 104 feeds the corrected B-mode image signal to the DSC 48.

As described above, an ultrasound image of high accuracy can be generated with no distortion by taking an ultrasound image for distortion amount calculation using the light irradiation by the light irradiator 44, and correcting a B-mode image signal (ultrasound image for diagnosis) with the distortion amount Dᵢⱼ which is calculated from the positions of the lattice point Pᵢⱼ as irradiated with light and of the bright spot Sᵢⱼ as detected from the ultrasound image for distortion amount calculation. A more accurate diagnosis can be conducted on the region to be diagnosed in a subject by taking ultrasound images of high accuracy.

The present invention is basically as described above.

While detailed as above, the present invention is in no way limited to the above described embodiments. Various improvements and modifications may be made within the scope of the invention.

What is claimed is:

1. An ultrasound diagnostic apparatus comprising:
an ultrasound probe including a transducer array for transmitting ultrasound, receiving an ultrasonic echo reflected from a subject and outputting a reception signal in response to ultrasound received; and
a diagnostic apparatus body including an image generating section for generating an ultrasound image for diagnosis in accordance with the reception signal as outputted from the transducer array,
the apparatus further comprising:
a lattice point setting unit for setting lattice points in a region to be imaged;
a light irradiator for irradiating each of the lattice points as set by the lattice point setting unit with light converging thereto, to thereby impart heat locally to the irradiated lattice point;
an image for distortion amount calculation-generating section for generating an ultrasound image for distortion amount calculation based on a reception signal outputted from the transducer array having received ultrasound resulting from irradiation of an inside of the subject with light by the light irradiator;
a lattice point detector for detecting a position of the irradiated lattice point on the ultrasound image for distortion amount calculation; and
a distortion amount calculating section for calculating, as a distortion amount, difference between absolute coordinates of the lattice point as irradiated with light by the light irradiator and the position of the lattice point that is detected on the ultrasound image for distortion amount calculation.

2. The ultrasound diagnostic apparatus according to claim 1, wherein:
said transducer array transmits ultrasound to said subject during the irradiation of the inside of the subject with light by said light irradiator;
the transducer array receives not only ultrasound resulting from the irradiation of the inside of the subject with light by the light irradiator but an ultrasonic echo resulting from transmission of ultrasound to the subject by the transducer array, so as to output a reception signal; and
said image for distortion amount calculation-generating section generates said ultrasound image for distortion amount calculation based on a reception signal outputted from the transducer array.

3. The ultrasound diagnostic apparatus according to claim 1, further comprising an image corrector for correcting said ultrasound image for diagnosis using said distortion amount calculated by said distortion amount calculating section.

4. The ultrasound diagnostic apparatus according to claim 1, wherein said light irradiator is provided on said ultrasound probe.

5. The ultrasound diagnostic apparatus according to claim 1, further comprising a sound speed map generating section for generating a sound speed map in said subject, wherein:
the sound speed map generating section uses said distortion amount calculated by said distortion amount calculating section to correct said reception signal assigned to sound speed map generation, so as to generate the sound speed map.

6. The ultrasound diagnostic apparatus according to claim 5, wherein the image generating section for generating an ultrasound image generates the ultrasound image using the sound speed map as generated by said sound speed map generating section.

7. The ultrasound diagnostic apparatus according to claim 6, wherein the sound speed map as generated by said sound speed map generating section is displayed in such a manner that it is superimposed on the ultrasound image as generated by said image generating section.

8. The ultrasound diagnostic apparatus according to claim 1, wherein said light irradiator has a light source array including a plurality of light sources, and a microlens array including a plurality of microlenses associated with the light sources of the light source array, respectively.
